(19) United States
(12) Patent Application Publication
(10) Pub. No.: US 2003/0153617 A1
(43) Pub. Date: Aug. 14, 2003

(54) Simvastatin dosage forms

(76) Inventors: Frans Van Dalen, Nijmegen (NL); Jacobus M. Lemmens, Mook (NL)

Correspondence Address: FLESHNER & KIM, LLP, P.O. BOX 221200, CHANTILLY, VA, 20153, US

(21) Appl. No.: 10/320,746

(22) Filed: Dec. 17, 2002

Related U.S. Application Data
(60) Provisional application No. 60/340,268, filed on Dec. 18, 2001.

(51) Int. Cl. A61K 31/366

(52) U.S. Cl. 514/460

(57) ABSTRACT

A pharmaceutical tablet comprising an effective amount of simvastatin provides several advantages including reliable or improved storage stability by having a pH within the range of 5.0 to 7.5, based on a 20 wt % aqueous slurry.

This application claims the benefit of priority under 35 U.S.C. §119(e) from provisional patent application Ser. No. 60/340,268, filed Dec. 18, 2001, the entire contents of which are incorporated herein by reference.

### BACKGROUND OF THE INVENTION

The present invention relates to pharmaceutical tablet compositions containing simvastatin and to methods of making and using the same.

U.S. Pat. No. 4,444,784 describes an antihypercholesterolemic agent having the following structure (1):

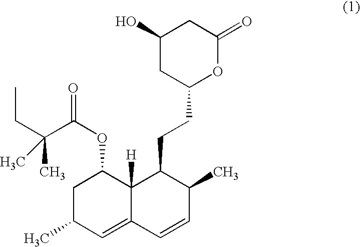

which is now known as simvastatin. Simvastatin is an effective 3-hydroxy-3-methylglutaryl-coenzyme A (HMG-CoA) reductase inhibitor. The inhibition of HMG-CoA reductase causes a reduction in cholesterol production and hence the antihypercholesterolemic effect. Simvastatin is commercially sold in the U.S. and elsewhere under the brand name ZOCOR™ by Merck & Company, Inc. In general, the commercially available ZOCOR™ tablets contain simvastatin (5, 10, 20, 40, or 80 mg), anhydrous lactose, microcrystalline cellulose (carriers), pregellatinized maize starch (disintegrant), magnesium stearate (lubricant), butylated hydroxyanisol (BHA), citric acid monohydrate and ascorbic acid (antioxidants). The tablet is coated by a water-dispersible film-coat comprising hydroxypropyl cellulose, HPMC, talc and colorants.

Simvastatin is a prodrug in that it is converted in vivo to the corresponding active, ring open, hydroxy acid form of the following structure (2):

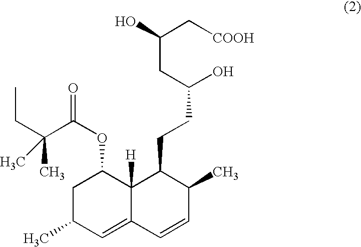

The lactone ring form is lipophilic and it is believed to provide selective accumulation in the liver where it is efficiently metabolized to the much less lipophilic active hydroxy acid form. See Hamelin and Turgeon, TiPS, Vol. 19, January 1998, pp 26-37, “Hydrophilicity/lipophilicity: relevance for the pharmacology and clinical effects of HMG-CoA reductase inhibitors.” The hydroxy acid form, while being active, is apparently more difficult to be absorbed, hence the use of the lactone prodrug form.

The lactone ring and hydroxy acid forms of simvastatin may exist in equilibrium. In general, it is believed that the lactone form of simvastatin predominantly exists under acidic conditions, while the hydroxy acid form is preferably formed under alkaline conditions.

Presumably the presence of ascorbic and citric acids in the commercially available tablets insures that the microenvironmental pH is acidic, thereby stabilizing the simvastatin in the preferred lactone form. These acids, along with BHA, a weak acid, also serve as antioxidants and thus may also be present to insure that the double bonds in the ring structure do not react; i.e. do not form epoxides. Because of the acid content, the commercially available ZOCOR™ 5 mg and 40 mg tablets, for instance, exhibit a pH of about 2.7-2.9 (measured in a 20% aqueous slurry).

However, the commercially available simvastatin tablets suffer from several disadvantages. First, the in vitro dissolution rate of the tablets can substantially alter during storage. For instance, one sample obtained commercially of simvastatin tablets (ZOCOR™ 40 mg) showed an initial dissolution rate of 98% in 30 minutes but only 13% in 30 minutes after 6 months accelerated storage in the original package at 40° C./75% RH. The dissolution was measured by USP method II (paddle) using an aqueous sodium hydrogen phosphate solution containing 0.5% of sodium lauryl sulphate with the pH adjusted to pH 7.0 by 5 M sodium hydroxide. Similar decreases in dissolution were also observed for ZOCOR™ 5 mg tablets.

Second, the appearance of the tablet composition changes substantially during such a storage period. The tablet cores tend to turn creamy to light brown, most probably due to oxidation of oxygen-sensitive acidic excipients present in the composition. The discoloration becomes apparent much sooner than does the decrease in dissolution.

Accordingly, care must be used in storing the commercial tablet to avoid high temperatures and/or high humidity. Exposure to these conditions can alter the tablet including its release characteristics. It would be desirable to have a simvastatin composition that did not suffer from the threat of the above-mentioned stability issues.

Further, the ZOCOR™ tablet is believed to be made by a wet granulation process in view of how well the acids are dispersed and in view of the water content; generally 5 to 6%. It would also be desirable to make a tablet by dry methods. A dry process for making tablets is generally more economical, but can have mixing or reproducibility issues.

### SUMMARY OF THE INVENTION

The present invention is based on the discovery that a stable simvastatin pharmaceutical composition can be formed when the pH is within the range of about 5.0 to 7.5. Accordingly, a first aspect of the present invention relates to a pharmaceutical tablet core composition comprising an effective amount of simvastatin and at least one pharmaceutically acceptable excipient, wherein said composition exhibits a pH within the range of 5.0 to 7.5. The tablet core can be used uncoated or can be coated to form a coated tablet; i.e. a film coated tablet. The tablet typically contains a sugar and/or a cellulose as the main excipient(s) and generally also contains a lubricant, a disintegrant, and an antioxidant, but is not limited thereto. In a preferred embodiment, the antioxidant is BHA and more preferably no strong acidic antioxidant is used, especially no ascorbic acid and/or citric acid.

The tablet core composition preferably has a low water content, i.e., 2 wt % or less, and is more preferably a dry made tablet. Surprisingly, a uniform tablet can be made without water, even when small amounts of one or more excipients are used. For instance, an antioxidant such as BHA is normally contained in small amounts, typically less than 0.1%, and yet need to be uniformly dispersed throughout the tablet. This can be achieved, preferably by the use of progressive mixing in a dry mixing process.

Various embodiments of the present invention yield a tablet that is more stable than the commercial simvastatin tablets but is nonetheless bioequivalent to the commercial tablet and this represents a particularly preferred aspect of the present invention. The blended, pre-compressed composition having a pH of 5.0 to 7.5 can also be used to fill capsules which is another embodiment of the present invention.

### DETAILED DESCRIPTION OF THE INVENTION

The present invention relates to tablets that contain an effective amount of simvastatin and at least one pharmaceutically acceptable excipient wherein the pH of the composition is within the range of 5.0 to 7.5. Preferably, the pH is within the range of 5.0 to less than 7.0, such as 5.5 to 6.7, more typically 5.5 to 6.5, still more typically 6.0 to 6.5 or 6.1 to 6.4. As used herein, the pH of the tablet composition refers to the pH as determined by forming a slurry of the solid composition with water and measuring the pH of the slurry, as is understood by workers skilled in the art regarding the pH of a solid composition. The concentration of the composition in the slurry is 20 wt %. The pH is measured by any standard technique.

Simvastatin is a well known antihypercholesterolemic agent and can be made by techniques known in the art. See U.S. Pat. Nos. 4,444,874, 4,820,850, U.S. Pat. No. 5,393,893, 6,100,407, and 6,271,398. The simvastatin contained in the tablets of the present invention is mostly in the lactone ring form; i.e., at least 90%, typically at least 97%, more typically at least 98%, and preferably at least 99.5% of the simvastatin is in the lactone form with the remainder being the ring open hydroxy acid form. The amount of lactone versus hydroxy acid form can be determined in a variety of ways by using and applying techniques that are well known in the art. Advantageously, HPLC is used to determine this ratio, if desired.

The amount of simvastatin present in the tablet composition is effective to inhibit HMG-CoA reductase and thus be useful in treating any condition where suppression of cholesterol would be beneficial. For example, simvastatin can be used in an amount effective to treat atherosclerosis or hyperlipemia. Generally the amount of simvastatin in a tablet ranges from 1 to 500 mg, more usually 2 to 200 mg. Preferred embodiments contain 5, 10, 20, 30, 40, 50, 80 mg, or 160 mg of simvastatin.

The tablet compositions of the present invention also contain at least one excipient. An “excipient” as used herein means any pharmaceutically acceptable inactive component of the composition. As is well known in the art, excipients include diluents, binders, lubricants, disintegrants, colorants, antioxidants/preservatives, pH-adjusters etc. The excipients are selected based on the desired physical aspects of the final form: e.g., obtaining a tablet with desired hardness and friability, being rapidly dispersible and easily swallowed, etc. The desired release rate of the active substance from the composition after its ingestion also plays a role in the choice of excipients.

Suitable excipients for use in this invention include:

a diluent such as calcium hydrogen phosphate, lactose, mannitol etc.

a binder such as microcrystalline cellulose or a modified cellulose, povidone etc.

a disintegrant such as sodium starch glycollate, crosspovidone

a lubricant such as magnesium stearate, sodium stearyl fumarate, talc

a colorant, taste masking agent, etc.

The tablet core composition typically contains at least a diluent and optionally a binder. The diluent is preferably a sugar such as lactose or mannitol, more preferably lactose anhydrate or a calcium phosphate such as dibasic calcium phosphate dihydrate, dibasic calcium phosphate anhydrate, etc. The binder is typically a cellulose such as microcrystalline cellulose or a modified cellulose. Generally the tablet core contains a diluent as the majority constituent of the composition; i.e. at least 55 wt %, preferably at least 60 wt %

The tablet core composition generally contains an antioxidant, preferably BHA, more preferably BHA is the only antioxidant. In some embodiments, BHA is used in the absence of any strong acid antioxidants such as ascorbic acid, citric acid, etc. The amount of BHA is generally 0.1 wt % or less, more typically 0.01 to 0.05 wt %.

The tablets of the present invention preferably contain low amounts of water, preferably 2% or less total water. This includes any water that is bound, as is conventional for microcrystalline cellulose, as well as free water. This low water content is typically only achieved in a dry made tablet. A pharmaceutical tablet is “dry made” for purposes of the present invention if the simvastatin active material is combined with/dispersed in the excipient(s) without the use of a liquid. Generally, a dry made tablet has a different structure under scanning electron microscope examination than a comparable wet made tablet, especially a wet granulation made tablet, and can thus be identified as such without actually witnessing the method of manufacture. Thus, while the manufacturing technique is dispositive on whether the tablet was dry made, a person skilled in the art can usually determined that a tablet was dry made, or not, by examination.

The tablet can be uncoated which is also referred to herein as a tablet core. A tablet core can be used as a pharmaceutical composition. The tablets may be covered with a suitable coating. For example, the coating can be a moisture or light barrier to help with storage stability. Alternatively, the coating may provide release control properties such as an enteric coat. The coating usually has little to no effect on the pH of the composition. Accordingly, the tablet core as well as the coated tablet will normally exhibit the same pH. However, in some embodiments, only the uncoated tablet core will satisfy the above pH range.

The tablets can be packaged in appropriate pharmaceutically acceptable bottles or in blister packs.

The tablets can be immediate release tablets, modified release, extended release, or delayed release tablets. Preferably the tablets are immediate release tablets and more preferably the release rate or release curve is comparable or bioequivalent to commercially available simvastatin tablets (ZOCOR™). An “immediate release” tablet is one wherein at least 90% of the simvastatin is released in 30 minutes from start of a dissolution test. The “dissolution test” for purposes of the present invention uses the USP apparatus II (paddles) and an aqueous sodium hydrogen phosphate solution that further contains 0.5% of sodium lauryl sulphate and is adjusted to pH 7.0 by 5 M sodium hydroxide as the dissolution media. The surfactant is present due to the low water solubility of simvastatin.

The tablets of the present invention preferably exhibit sufficient stability that after storage for six months in an environment of 40° C. and 75% relative humidity (RH), the tablet does not change its dissolution result at 30 minutes by more than 10%, preferably not more than 5%. Preferred tablets will exhibit at least 90% release of the simvastatin at 30 minutes from the start of the dissolution test, even after six-month storage at 40° C./75% RH.

The pharmaceutical compositions of the present invention can be made by techniques generally known in the art. In general, the simvastatin is mixed with one or more excipients to form a blend. The mixing can be carried out wet or dry (i.e. using or not using a solvent or a liquid diluent in the process) and can involve granulating, slugging, or blending of powders. A dry process is preferred as mentioned above. The blend, after optional further processing, can be compressed into tablets.

In a preferred process, BHA is blended with a pharmaceutically acceptable excipient such as a portion of the microcrystalline cellulose to form a first blend. This first blend is then subjected to an additional blending step or steps wherein one or more additional excipients and the simvastatin are added to the blend to form a precompression blend. The precompression blend is then compressed to form tablets.

Additional processes can be performed between these steps or in conjunction therewith. For example, milling is preferably performed after the first blend is formed and before additional excipient(s) is added. Milling can be done at any time in the process including before the first blending step, after the first blending step, before or during the additional blending step or steps and after the additional blending step(s). The blending itself may be progressive in that a portion of the excipient is added, blended, more excipient added, further blended, etc. Furthermore, sieving can be performed as needed; i.e., when agglomerates are formed and desired to be removed.

Dry granulation, also known as compaction, can also be used in conjunction with this process. Specifically, any blend containing the simvastatin can be compacted and then milled to form a second blend. Additional excipient(s) in terms of amount or kind can be added to the second blend and blended therein. Alternatively, the second blend can be the final precompression blend that is ready for compression into tablets.

Once compressed into a tablet, the cores may optionally be coated by conventional techniques.

While a tablet is preferred, it is possible to use any of the above blends such as the precompression blend to fill a capsule and thus form a capsule dosage form.

The tablets of the present invention are useful in treating various conditions that would be benefited by a reduction in the production of cholesterol, including but not limited to treating hyperlipidemia and/or atherosclerosis. The tablets of the present invention are administered to a patient in need thereof so as to provide an effective antihypercholesterolemic amount of simvastatin. The tablets can be taken once a day, several times a day, or two or more together at one or more times per day. Preferably one tablet is a unit dose and hence only one tablet is taken per administration.

The invention will be further illustrated by way of the following non-limiting example.

### EXAMPLES 1-4

Tablets according to the present invention and containing the ingredients and amounts (per tablet) as shown below were made according to the following general process:

BHA and MCC were mixed in a ratio of 1:10.

The remaining MCC, the simvastatin and the lactose were mixed with the pre-mixed BHA and MCC.

The resulting blend was compacted to increase the density and particle size to form granules (dry granulation).

The magnesium stearate and talc were mixed with the granules for 5 minutes.

The blend was compressed into tablets and the tablets film coated.

| Ingredients | Example 1 | Example 2 | Example 3 | Example 4 |
|---|---|---|---|---|
| Core composition | | | | |
| Simvastatin | 5 mg | 10 mg | 20 mg | 40 mg |
| Lactose anhydrate | 79.50 mg | 74.50 mg | 149.00 mg | 298.0 mg |
| Microcrystalline | 5.0 mg | 5.0 mg | 10.0 mg | 20.0 mg |
| cellulose (MCC) | | | | |
| Pregelatinised Maize | 10.0 mg | 10.0 mg | 20.0 mg | 40.0 mg |
| starch | | | | |
| BHA | 0.02 mg | 0.02 mg | 0.04 mg | 0.08 mg |
| Mg Stearate | 0.50 mg | 0.50 mg | 1.00 mg | 2.00 mg |
| Talc | 1.00 mg | 1.00 mg | 2.00 mg | 4.00 mg |
| Coating composition | | | | |
| Hydroxypropyl | 0.76 mg | 0.76 mg | 1.65 mg | 3.06 mg |
| cellulose | | | | |
| Methylhydroxypropyl | 0.76 mg | 0.76 mg | 1.65 mg | 3.06 mg |
| cellulose | | | | |
| Talc | 0.28 mg | 0.28 mg | 0.60 mg | 1.11 mg |
| Titanium dioxide | 0.69 mg | 0.69 mg | 1.50 mg | 2.78 mg |
| Iron oxide yellow | 0.05 mg | — | — | — |

For comparison the composition of the commercially available ZOCOR™ tablets is set forth below:

| Ingredients | Commercial tablet | Commercial Tablet |
|---|---|---|
| Core composition | 10 mg | 20 mg |
| Simvastatin | 10 mg | 20 mg |
| Lactose anhydrate | 70.70 mg | 141.50 mg |
| Microcrystalline | 5.0 mg | 10.0 mg |
| cellulose (MCC) | | |
| Pregelatinised Maize | 10.0 mg | 20.0 mg |
| starch | | |
| BHA | 0.02 mg | 0.04 mg |
| Mg Stearate | 0.50 mg | 1.00 mg |
| Talc | 1.00 mg | 2.00 mg |
| Ascorbic acid | 2.5 mg | 5.0 mg |
| Citric acid | 1.25 mg | 2.5 mg |
| Coating composition | | |
| Hydroxypropyl | 0.76 mg | 1.65 mg |
| cellulose | | |
| Methylhydroxypropyl | 0.76 mg | 1.65 mg |
| cellulose | | |
| Talc | 0.28 mg | 0.60 mg |
| Titanium dioxide | 0.69 mg | 1.50 mg |
| Iron oxide yellow | 0.0015 mg | 0.092 mg |
| Iron oxide red | 0.0038 mg | 0.023 mg |

### Stability Example

A batch of tablets of the present invention similar to Examples 1 and 4 above were made and a sample portion thereof subjected to dissolution testing. Another sample portion was placed in a controlled environment of 40° C. and 75% RH for six months and then subjected to dissolution testing. The amount of simvastatin released at the 30 minute point of the test is reported below. For comparison, commercially obtained 5 mg and 40 mg ZOCOR™ tablets were also subjected to the same test and storage conditions and the amount of simvastatin released at the 30 minute point is also reported below.

| | Before Storage | After Storage |
|---|---|---|
| | amount of simvastatin | amount of simvastatin |
| | released at 30 minutes | released at 30 minutes |
| Inventive tablets | 95% | 94% |
| 40 mg strength | | |
| ZOCOR ™ 40 mg | 98% | 13% |
| Inventive tablets | 95% | 92% |
| 5 mg strength | | |
| ZOCOR ™ 5 mg | 98% | 12% |

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

What is claimed is:

1. A pharmaceutical tablet composition comprising an effective amount of simvastatin and at least one pharmaceutically acceptable excipient, wherein said composition exhibits a pH within the range of 5.0 to 7.5.

2. The composition according to claim 1, wherein said composition exhibits a pH within the range of 5.0 to less than 7.0.

3. The composition according to claim 2, wherein said composition exhibits a pH within the range of 5.5 to 6.7.

4. The composition according to claim 3, wherein said composition exhibits a pH within the range of 6.0 to 6.5.

5. The composition according to claim 4, wherein said composition exhibits a pH within the range of 6.1 to 6.4.

6. The composition according to claim 1, wherein said composition has a water content of 2% by weight or less.

7. The composition according to claim 1, which further comprises BHA.

8. The composition according to claim 7, wherein said composition does not contain ascorbic acid.

9. The composition according to claim 1, which comprises a sugar or a calcium phosphate as a diluent.

10. The composition according to claim 9, wherein said diluent is anhydrous lactose.

11. The composition according to claim 9, which further comprises a cellulose.

12. The composition according to claim 11, wherein said cellulose is microcrystalline cellulose.

13. The composition according to claim 1, which comprises anhydrous lactose, microcrystalline cellulose, a disintegrant, a lubricant, and BHA.

14. The composition according to claim 13, wherein said lactose provides at least 55% by weight of said composition.

15. The composition according to claim 1, wherein said tablet core is dry made.

16. The composition according to claim 1, wherein said composition exhibits release of at least 90% of the simvastatin in 30 minutes of a dissolution test.

17. The composition according to claim 16, wherein said composition exhibits stability under six month accelerated storage conditions of 60° C. and 75% RH such that the amount of simvastatin released in 30 minutes of a dissolution test of the stored composition is not more than 5% different than the amount released in 30 minutes of the pre-stored composition.

18. The composition according to claim 1, which further comprises a film coat thereon.

19. The composition according to claim 1, wherein said tablet is uncoated.

20. The composition according to claim 1, wherein said amount of simvastatin is selected from the group consisting of 5, 10, 20, 30, 40, 50, 80, and 160 mg.
